(19) United States
(12) Patent Application Publication
(10) Pub. No.: US 2008/0114348 A1
(43) Pub. Date: May 15, 2008

(54) Cryoprotective Agent Delivery

(76) Inventors: David W. Vancelette, San Diego, CA (US); Douglas A. Devens, Highland Park, IL (US)

Correspondence Address: AMS RESEARCH CORPORATION, 10700 BREN ROAD WEST, MINNETONKA, MN, 55343, US

(21) Appl. No.: 11/939,180

(22) Filed: Nov. 13, 2007

Related U.S. Application Data
(60) Provisional application No. 60/865,555, filed on Nov. 13, 2006.

(51) Int. Cl. A61B 18/02

(52) U.S. Cl. 606/23

(57) ABSTRACT

The present disclosure is directed to the use of a cryoprotective agent to protect healthy tissue while damaged tissue is cryosurgically treated. A cryoprotective agent can be delivered utilizing a delivery probe that is inserted into healthy tissue either prior to or during a freeze portion of a first freeze/thaw cycle. The cryoprotective agent diffuses through the healthy tissue, but diffusion is controlled at the freeze edge by the diffusion limiting characteristics of the tissue that is frozen. When the frozen tissue is thawed, the disrupted tissue will continue to restrict the diffusion of the cryoprotective agent. Additional freeze/thaw cycles can then be conducted and the cryoprotective agent will continue to protect the healthy tissue. The delivery probes can also function as thermal sensor probes that can also be used to monitor the temperature at selected tissue locations.

### PRIORITY CLAIM

The present application claims priority to U.S. Provisional Application Ser. No. 60/865,555, filed Nov. 13, 2006 and entitled “CRYOPROTECTIVE AGENT DELIVERY”, which is herein incorporated by reference in its entirety.

### FIELD OF THE INVENTION

The present disclosure relates to cryosurgical systems for use in the treatment of benign or cancerous tissues, and more particularly to use of a cryoprotective agent in a cryosurgical system.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryosurgical treatment can be used to treat prostate cancer and benign prostate disease. Cryosurgery also has gynecological applications. In addition, cryosurgery may be used for the treatment of a number of other diseases and conditions including, but certainly not limited to, breast cancer, liver cancer, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

However, there is a risk that during cryosurgery healthy tissues, nerves, or blood vessels surrounding targeted regions can be accidentally frozen. When the cryosurgical procedure involves treatment of prostate cancer, accidental freezing of surrounding tissue, nerves and blood vessels can lead to complications include erectile dysfunction, incontinence and/or impotence.

### SUMMARY OF THE INVENTION

The present disclosure is directed to the use of a cryoprotective agent to protect healthy tissue that surrounds targeted, damaged tissue during a cryosurgical procedure. In order to protect the surrounding healthy tissue, a cryoprotective agent can be introduced to the surrounding healthy tissue. Generally, the cyroprotective agent is locally introduced utilizing a delivery probe that can administer the cyroprotective agent into healthy tissue either prior to or during a freeze portion of a first freeze/thaw cycle. In the case of cryosurgical treatment of prostate cancer, the cryoprotective agent can be locally introduced into the neurovascular bundles using pre-inserted delivery probes. The cryoprotective agent diffuses through the healthy tissue but fails to penetrate the targeted, damaged tissue by the diffusion limiting characteristics resulting from freezing of the targeted, damaged tissue. The freezing of the targeted, damaged tissue damages and disrupts the tissue and vasculature that is frozen preventing diffusion of the cyroprotective agent into the targeted, damaged tissue. When the frozen tissue is thawed, the targeted, damaged tissue remains disrupted and continues to restrict the diffusion of the cryoprotective agent. Additional freeze/thaw cycles can then be conducted and the cryoprotective agent will continue to protect the healthy tissue, while it remains prevented from diffusing into the damaged, targeted tissue. In some representative embodiments, the delivery probes can additionally function as thermal sensor probes that can also be used to monitor the temperature at selected tissue locations surrounding the targeted, damaged tissue.

In one aspect of the present disclosure, a cryosurgical system utilizes a cryoprotective agent to protect healthy tissue from being accidentally frozen during a cryosurgical procedure. Prior to or during the freezing of damaged tissue with a cryoprobe, a delivery probe can be inserted into the healthy tissue. Once inserted, the delivery probe can release a cryoprotective agent that diffuses into the healthy tissue. Multiple freeze/thaw cycles can then be conducted with the cryosurgical system as the cryoprotective agent continues to protect the healthy tissue throughout the cryosurgical treatment procedure. The cryosurgical system can provide for especially advantageous results in the cryosurgical treatment of prostate cancer.

In another aspect of the present disclosure, a method of performing cryosurgery on damaged tissue utilizes a cryoprotective agent to protect adjacent, healthy tissue. Prior to or during a first freeze cycle, delivery probes are inserted into the healthy tissue. Once inserted, the delivery probes release a cryoprotective agent into the tissue. While the cryoprotective agent diffuses through the healthy tissue, the ice ball formed on the cryoprobe tip grows to the edge of the area targeted for freezing. The cryoprotective agent diffuses until it covers all of the healthy tissue, but does not diffuse into the frozen tissue region. When the cryoprotective agent has finished diffusing through the healthy tissue, additional damaged tissue can optionally be frozen, and then the frozen tissue can be thawed. After thawing, the previously frozen tissue remains disrupted and continues to prevent diffusion of the cryoprotective agent. Additional freeze/thaw cycles can then be conducted while the cryoprotective agent protects the healthy tissue. After the procedure, the cryoprotective agent can be removed using negative pressure. In some embodiments, the method of performing cryosurgery is especially effective in treating prostate cancer.

In another aspect of the present disclosure a cryosurgical delivery probe can protect healthy tissue located in proximity to targeted tissue during a cryosurgical treatment procedure. The cryosurgical delivery probe can comprise an amount of a cryoprotective agent that is delivered into the healthy tissue. In some embodiments, the cryosurgical delivery probe can further comprise a temperature sensor for relaying temperature information to a cryosurgical treatment system during a cryosurgical treatment procedure.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a cryosurgical system according to the present disclosure.

FIG. 2 is a side view of a cryosurgical prostate treatment utilizing a cryosurgical treatment system according to the present disclosure.

FIG. 3 is a flowchart of the process steps of an embodiment of a cryosurgical procedure using a cryoprotective agent according to the present disclosure.

### DETAILED DESCRIPTION

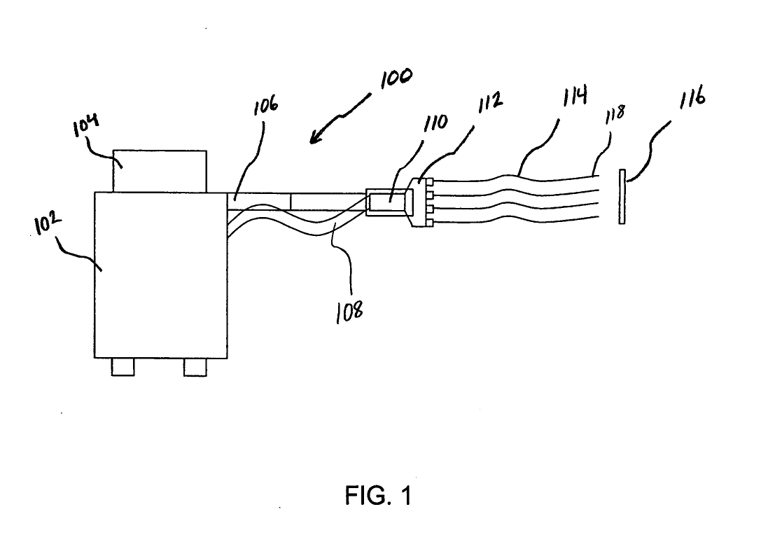

A closed loop cryosurgical system 100 according to the present disclosure is depicted in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. The use of mixed gas refrigerants is generally known in the art to provide a dramatic increase in cooling performance over the use of a single gas refrigerant. Control console 102 can also include controls that allow for the activation, deactivation, and modification of various system parameters, such as, for example, gas flow rates, pressures, and temperatures of the mixed gas refrigerants. Display 104 can provide the operator the ability to monitor, and in some embodiments, adjust the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

With reference to FIG. 1, the high pressure primary refrigerant is transferred from control console 102 to a cryostat heat exchanger module 110 through a flexible line 108. The cryostat heat exchanger module 110 can include a manifold portion 112 that transfers the refrigerant into and receives refrigerant out of one or more cryoprobes 114. Each cryoprobe includes a tip portion 118 that is cooled by the refrigerant and used to freeze tissue during a cryosurgical procedure. The cryostat heat exchanger module 110 and cryoprobes 114 can also be connected to the control console 102 by way of an articulating arm 106, which can be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106. A positioning grid 116 can be used to properly align and position the cryoprobes 114 for patient insertion.

Cryosurgery often involves a cycle of treatments in which the targeted tissue is frozen, allowed to thaw, and then refrozen. Double and even triple freeze/thaw cycles are now commonly used in cryosurgery. Comparison with a single freeze/thaw cycle shows that additional freeze/thaw cycles can increase the damage to the targeted tissue, thus providing for a more beneficial and efficacious treatment.

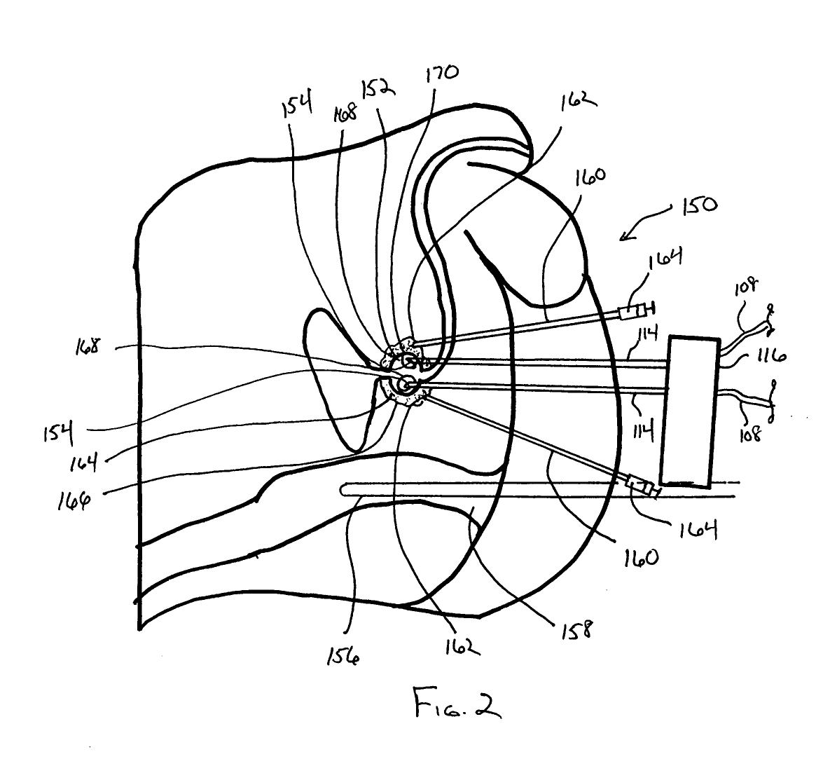

As illustrated in FIG. 2, a patient undergoing cryosurgical treatment for prostate cancer is illustrated in a traditional lithotomy position 150. Positioning grid 116 is proximate the patient such that cryoprobes 114 can be inserted into the prostate 152. Tip portion 118 of cryoprobes 114 can comprise a trocar configuration 154 allowing for insertion and penetration into prostate 152. A medical imaging sensor 156 such as, for example, a transrectal ultrasound probe can be positioned within rectum 158 to guide the insertion of the cryoprobes 114. In addition, one or more delivery probes 160 can be positioned such that a dispensing tip 162 is located proximate a portion of prostate 152 to be treated. Delivery probes 160 allow a cyroprotective agent 164 to be introduced into surrounding tissue 166.

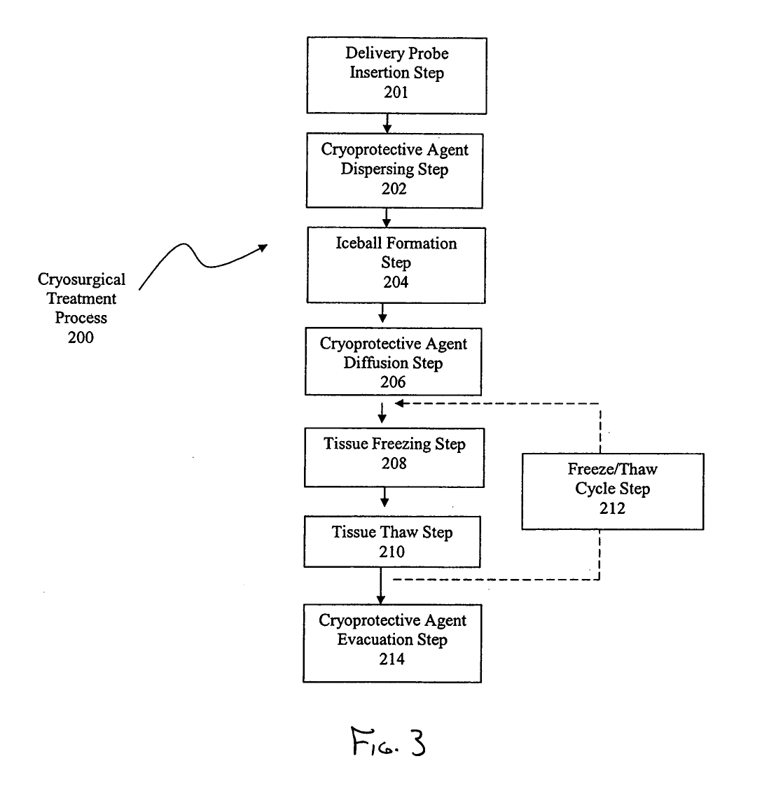

Referring to FIG. 3, there can be seen a flowchart illustrating a cryosurgical treatment process 200 that can be implemented to protect healthy surrounding tissue 166 with a cryoprotective agent 164 during a cryosurgical procedure as previously shown in FIG. 2. Healthy tissue that it may be desirable to protect can include, for example, neurovascular bundles, denonvilliers fascia, the urethra, and the rectum. To avoid undesirable freezing of such areas, delivery probes 160 containing cryoprotective agent 164 can be inserted into healthy tissue adjacent the targeted tissue prior to freezing at a delivery probe insertion step 201. Once the delivery probes 160 are inserted, the cryoprotective agent 164 can be released and begin to diffuse through the surrounding tissue 166 at a cryoprotective agent dispersing step 202. Representative delivery probes 164 can comprise needle-based syringe-style delivery probes or high pressure, needleless injection systems utilizing high pressure delivery lumens.

Referring again to FIGS. 2 and 3, an iceball 168 formed on the tip 118 grows to the edge of the targeted zone, or freeze edge 170, at an iceball formation step 204 and can remain in that position for the time required for the cryoprotective agent 164 to diffuse to the freeze edge at a cryoprotective agent diffusion step 206. Where necessary, cryoprobe freezing can be pulsed on and off to maintain proper iceball position and size during iceball formation step 204 until diffusion is completed during cryoprotective agent diffusion step 206. In some embodiments, the temperature of the cryoprotective agent 164 can be elevated prior to injection into the healthy surrounding tissue 166 in order to increase the rate of diffusion during cryoprotective agent diffusion step 206. In treatment locations where the cryoprotective agent 164 would diffuse to the freeze edge 170 prior to completion of the first freeze cycle, such as, for example, where there is a small area of healthy surrounding tissue 166 through which diffusion would occur and/or where there is a large area to be frozen, the delivery probes 160 can instead by inserted at a selected point during the freezing process, rather than before freezing is begun.

In some embodiments, the delivery probe 160 can additionally function as a thermal sensor probe. Thermal sensor probes are well known in the art and are generally used to read the temperature at selected tissue locations during a cryosurgical procedure. A thermal sensor probe can contain a cryoprotective agent 164 and release the cryoprotective agent 164 into the surrounding tissue 166 upon insertion. By incorporating the delivery of a cryoprotective agent 164 into a delivery probe 160 that further includes a thermal sensor probe, the cryosurgical procedure is improved by reducing the amount of equipment that must be used throughout the procedure.

Referring again to FIG. 3, once iceball formation step 204 and cryoprotective agent diffusion step 206 are complete, a tissue freezing step 208 can be initiated. During the tissue freezing step 208, the iceball 168 can be expanded beyond the freeze edge 170 to freeze additional targeted tissue adjacent the healthy surrounding tissue 166. The cryoprotective agent 164 will protect the healthy surrounding tissue 166 in the protective zone defined by the diffused cryoprotective agent 164, from being damaged by the expanded iceball 168. Once all desired tissue has been frozen, a tissue thaw step 210 can be initiated. When the tissue thaw step 210 is conducted, the previously frozen tissue will remain disrupted even when the previously frozen tissue is completely thawed, thereby preventing the cryoprotective agent 164 from diffusing into the previously frozen tissue. Optionally, a second, and possibly third, freeze/thaw cycle step 212 can be conducted so as to freeze as much of the targeted tissue as desired, while the cryoprotective agent 164 continues to prevent freezing of the adjacent healthy surrounding tissue 166. Once the cryosurgical treatment is completed, the cryoprotective agent can be removed using negative pressure, such as, for example, with a syringe plunger, pump, or other suitable device, at a cryoprotective agent evacuation step 214.

Representative examples of suitable cryoprotective agents 164 that are presently contemplated for use with the cryosurgical system of the present disclosure include glycerol, propylene, glycol, DMSA, DMSO, AFP, glucose, VM3, VEG, or some combinations of these. Injecting one or more, either individually or in combination, of the above cryoprotective agents 164 prior to or during cryosurgery can lead to enhanced cryoinjury regions, faster treatment times, cryotreatment at higher (and therefore safer) temperatures, greater localization of cryodamage, and improved protection of regions not targeted for freezing.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A method of performing a cryosurgical procedure comprising:
delivering a cryoprotective agent into healthy tissue adjacent to targeted tissue, wherein a freeze edge is defined at the boundary of the healthy tissue and the targeted tissue;
forming an iceball at a tip portion of a cryoprobe;
allowing the cryoprotective agent to diffuse through the healthy tissue surrounding the freeze edge;
freezing the targeted tissue with the iceball, wherein the diffused cyroprotective agent prevents damage to the healthy tissue.
thawing the targeted tissue; and
removing the cryoprotective agent from the healthy tissue.

2. The method of claim 1, further comprising:
growing the iceball to the freeze edge when the cryoprotective agent is released such that the iceball does not contact the healthy tissue.

3. The method of claim 1, further comprising:
growing the iceball beyond the freeze edge to contact healthy tissue in which the cyroprotective agent has been diffused.

4. The method of claim 1, wherein delivering the cryoprotective agent into the healthy tissue comprises:
administering the cyroprotective agent with a delivery probe before the targeted tissue is frozen.

5. The method of claim 1, wherein delivering the cyroprotective agent into the healthy tissue comprises:
administering the cryoprotective agent with a delivery probe while the targeted tissue is being frozen.

6. The method of claim 1, further comprising:
refreezing the targeted tissue with the cryoprotective agent present in the healthy tissue; and
rethawing the targeted tissue with the cryoprotective agent present in the healthy tissue.

7. The method of claim 1, further comprising:
monitoring temperatures at selected locations beyond the freeze edge with a thermal sensor incorporated into the delivery probe.

8. The method of claim 1, further comprising:
elevating a cyroprotective agent temperature prior to administering the cryoprotective agent into the healthy tissue.

9. The method of claim 1, wherein the cryoprotective agent is selected from the group consisting of: glycerol, propylene, glycol, DMSA, DMSO, AFP, glucose, VM3, and VEG.

10. The method of claim 1, wherein removing the cryoprotective agent includes withdrawing the cyroprotective agent using a needle-based syringe plunger.

11. The method of claim 1, wherein removing the cryoprotective agent includes applying a vacuum.

12. The method of claim 1, further comprising:
monitoring iceball growth with a medical imaging system.

13. The method of claim 12, further comprising:
pulsing delivery of a refrigerant to the tip portion of the cryoprobe to control iceball growth.

14. The method of claim 1, wherein the targeted tissue comprises prostate tissue.

15. A closed loop cryosurgical system for treating prostate cancer comprising:
a console for supplying a refrigerant fluid;
one or more cryoprobes fluidly connected to the console and configured to be cooled by the refrigerant for performing a cryosurgical procedure, the one or more cryoprobes adapted for insertion into targeted prostate tissue, wherein recirculation of the refrigerant fluid at a tip portion of the cryoprobe results in formation of an iceball at the tip portion; and
one or more delivery probes containing a selectively releasable cryoprotective agent, the one or more delivery probes adapted for insertion into healthy surrounding tissue.

16. The system of claim 15, further comprising:
a medical imaging system for monitoring iceball formation within the targeted prostate tissue.

17. The system of claim 16, wherein the console pulses delivery of the refrigerant fluid to the one or more cryoprobes based on monitoring of the iceball formation.

18. The system of claim 15, wherein the cryoprotective agent is selected from the group consisting of: glycerol, propylene, glycol, DMSA, DMSO, AFP, glucose, VM3, and VEG.

19. The system of claim 15, wherein the tip portion comprises a trocar configuration.

20. The system of claim 15, wherein the one or more delivery probes comprises a needle-based, syringe style delivery probe.
